(19) United States
(12) Patent Application Publication
(10) Pub. No.: US 2024/0245285 A1
(43) Pub. Date: Jul. 25, 2024

(54) MEDICAL MANIPULATOR SYSTEM, METHOD FOR CONTROLLING MEDICAL MANIPULATOR, AND CONTROL DEVICE

(71) Applicant: OLYMPUS MEDICAL SYSTEMS CORP., Tokyo (JP)

(72) Inventors: Ryota YANAGAWA, Hachioji-shi (JP); Kosuke KISHI, Mitaka-shi (JP)

(73) Assignee: OLYMPUS MEDICAL SYSTEMS CORP., Tokyo (JP)

(21) Appl. No.: 18/584,645

(22) Filed: Feb. 22, 2024

Related U.S. Application Data
(63) Continuation of application No. PCT/JP2022/039138, filed on Oct. 20, 2022.
(60) Provisional application No. 63/281,796, filed on Nov. 22, 2021.
(60) Provisional application No. 63/314,579, filed on Feb. 28, 2022.

(51) Int. Cl. A61B 1/005

(52) U.S. Cl. CPC A61B 1/0057

(57) ABSTRACT

A medical manipulator system includes a medical manipulator having a bending portion and a wire that bends the bending portion, an actuator that bends the bending portion by driving the wire, a sensor that detects a tension of the wire; and a control device that controls the actuator, wherein the wire includes a pair of first wires that bend the bending portion in a first direction, and when a tension difference between the pair of first wires is larger than a first threshold value, the control device performs first tension control to drive at least one wire of the pair of first wires so that the tension difference is equal to or less than the first threshold value.

### CROSS-REFERENCE TO RELATED APPLICATIONS

The present application is a continuation application based on PCT Patent Application No. PCT/JP2022/039138, filed on Oct. 20, 2022, of which priority is claimed on U.S. Provisional Application No. 63/281,796, filed on Nov. 22, 2021, and U.S. Provisional Patent Application No. 63/314,579, filed on Feb. 28, 2022, the entire contents of which are hereby incorporated by reference.

### BACKGROUND

### Field of the Invention

The present invention relates to a medical manipulator system, a method for controlling a medical manipulator, and a control device.

### Description of Related Art

Conventionally, medical manipulator systems have been used for observing and treating the inside of hollow organs such as the gastrointestinal tract. In a medical manipulator system, an insertion portion or the like inserted into a hollow organ can be electrically driven to become bent. A user can control a bending motion of the insertion portion or the like from an operating part disposed outside the body.

Japanese Patent (Granted) Publication No. 3549434 (hereinafter referred to as Patent Document 1) and Japanese Patent (Granted) Publication No. 3233373 (hereinafter referred to as Patent Document 2) describe endoscopes that, when an external force is applied to a bending portion that is electrically driven, control the bending portion to which the external force is applied.

However, conventional medical manipulator systems (for example, electric endoscope systems) shown in Patent Document 1 and Patent Document 2 distinguish between control that actively drives the bending portion using electric power (active control) and control that passively drives the bending portion when an external force is applied (passive control), require addition of additional mechanisms or have difficulty seamlessly switching between the active control and the passive control.

### SUMMARY

In view of the above circumstances, an object of the present invention is to provide a medical manipulator system, a method for controlling a medical manipulator, and a control device that can appropriately handle control that actively drives a bending portion using electric power and control that drives the bending portion passively when an external force is applied.

In order to solve the above problems, the present invention proposes the following means.

A medical manipulator system according to a first aspect of the present invention includes a medical manipulator having a bending portion and a wire that bends the bending portion, an actuator that bends the bending portion by driving the wire, a sensor that detects a tension of the wire; and a control device that controls the actuator, wherein the wire includes a pair of first wires that bend the bending portion in a first direction, and when a tension difference between the pair of first wires is larger than a first threshold value, the control device performs first tension control to drive at least one wire of the pair of first wires so that the tension difference is equal to or less than the first threshold value.

According to the medical manipulator system, the method for controlling a medical manipulator, and the control device of the present invention, it is possible to appropriately handle control that actively drives the bending portion by electric power (active control) and control that passively drives the bending portion when external force is applied (passive control).

### BRIEF DESCRIPTION OF THE DRAWINGS

FIG. 1 is an overall view of a medical manipulator system according to a first embodiment.

FIG. 2 is a view showing an endoscope and an operation device of the medical manipulator system used by an operator.

FIG. 3 is a view showing an insertion portion of the endoscope.

FIG. 4 is a cross-sectional view of a part of a bending portion of the insertion portion.

FIG. 5 is an enlarged view of a joint ring of the bending portion in a region E shown in FIG. 4.

FIG. 6 is a cross-sectional view of the bending portion taken along line C1-C1 in FIGS. 4 and 5.

FIG. 7 is a view showing a first attachment and detachment portion before being mounted in a drive device of the medical manipulator system.

FIG. 8 is a view showing an up-down wire attachment and detachment portion before being mounted in the drive device.

FIG. 9 is a view showing the up-down wire attachment and detachment portion mounted in the drive device.

FIG. 10 is a functional block diagram of the drive device.

FIG. 11 is a perspective view of the operation device of the medical manipulator system.

FIG. 12 is a functional block diagram of an image control device of the medical manipulator system.

FIG. 13 is a control flowchart of a drive controller of a control device of the medical manipulator system.

FIG. 14 is a view showing the insertion portion inserted into the large intestine.

FIG. 15 is a view showing the bending portion that is bent.

FIG. 16 is a view showing an example of the bending portion in which a tension difference equal to or larger than a predetermined threshold value has occurred.

FIG. 17 is a view showing another example of the same bending portion in which a tension difference equal to or larger than a predetermined threshold value has occurred.

FIG. 18 is a view showing a first attachment and detachment portion before being mounted in a drive device in a medical manipulator system according to a second embodiment.

FIG. 19 is a view showing a first attachment and detachment portion (an up-down wire attachment and detachment portion) before being mounted in the drive device.

FIG. 20 is a view showing the first attachment and detachment portion (up-down wire attachment and detachment portion) mounted in the drive device.

FIG. 21 is an overall view of a medical manipulator system according to a third embodiment.

FIG. 22 is a view showing a bending portion of the medical manipulator system.

FIG. 23 is a view showing a first attachment and detachment portion before being mounted in a drive device of the medical manipulator system.

### DETAILED DESCRIPTION

### First Embodiment

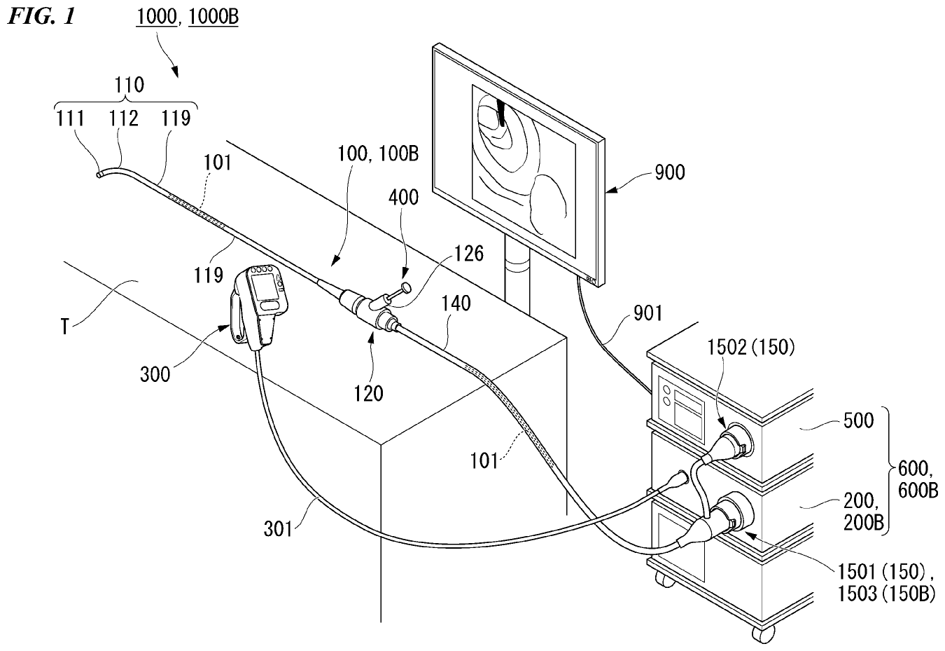

An electric endoscope system 1000 according to a first embodiment of the present invention will be described with reference to FIGS. 1 to 17. FIG. 1 is an overall view of the electric endoscope system 1000 according to this embodiment. The electric endoscope system 1000 is an example of a medical manipulator system. A medical manipulator includes an electrically driven endoscope, a catheter, a treatment tool, an endocrine device, and the like that are inserted into the body.

### Electric Endoscope System 1000

The electric endoscope system 1000, as shown in FIG. 1, is a medical system that observes and treats the inside of the body of a patient P lying on an operating table T. The electric endoscope system 1000 includes an endoscope 100, a drive device 200, an operation device 300, a treatment tool 400, an image control device 500, and a display device 900.

The endoscope 100 is a device that is inserted into a lumen of a patient P to observe and treat an affected area. The endoscope 100 is attachable to and detachable from the drive device 200. An internal path 101 is formed inside the endoscope 100. In the following description, the side of the endoscope 100 that is inserted into the lumen of the patient P will be referred to as the “distal end side A1,” and the side that is mounted in the drive device 200 will be referred to as the “proximal end side A2.”

The drive device 200 is detachably connected to the endoscope 100 and the operation device 300. The drive device 200 drives a built-in motor to electrically drive the endoscope 100 based on an operation input to the operation device 300. Further, the drive device 200 drives a built-in pump or the like to cause the endoscope 100 to perform air supply and suction based on an operation input to the operation device 300.

The operation device 300 is detachably connected to the drive device 200 via an operation cable 301. The operation device 300 may be able to communicate with the drive device 200 by wireless communication instead of wired communication. An operator S can electrically drive the endoscope 100 by operating the operation device 300.

The treatment tool 400 is a device that is inserted through the internal path 101 of the endoscope 100 and into the lumen of the patient P to treat the affected area. In FIG. 1, the treatment tool 400 is inserted into the internal path 101 of the endoscope 100 through a forceps port 126.

The image control device 500 is detachably connected to the endoscope 100 and acquires captured images from the endoscope 100. The image control device 500 causes the display device 900 to display captured images acquired from the endoscope 100 and GUI images and CG images for the purpose of providing information to the operator.

The drive device 200 and the image control device 500 constitute a control device 600 that controls the electric endoscope system 1000. The control device 600 may further include peripheral devices such as a video printer. The drive device 200 and the image control device 500 may be an integrated device.

The display device 900 is a device such as an LCD that can display images. The display device 900 is connected to the image control device 500 via a display cable 901.

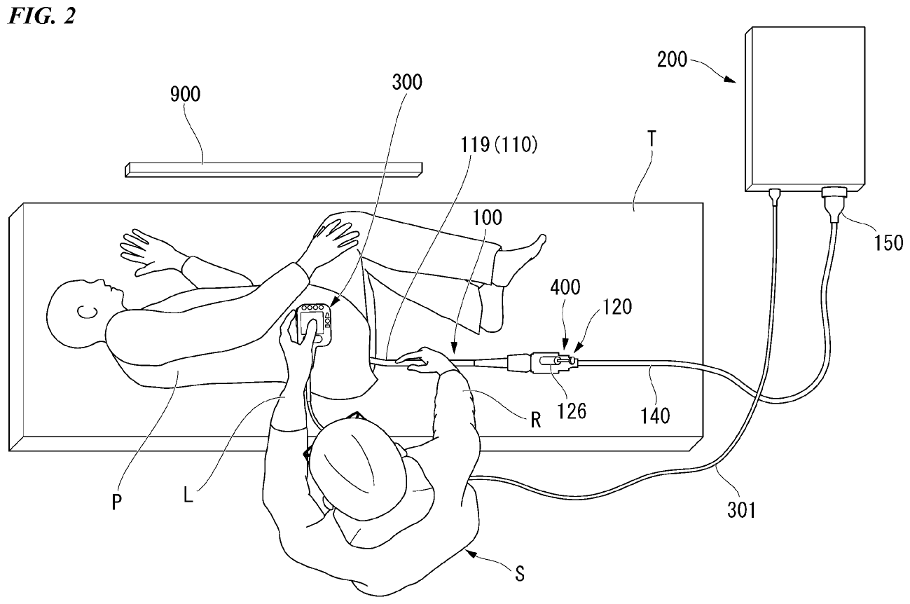

FIG. 2 is a view showing the endoscope 100 and the operation device 300 used by the operator S.

For example, the operator S operates the operation device 300 with the left hand L while observing the captured images displayed on the display device 900 and operating the endoscope 100 inserted into the lumen from the anus of the patient P with the right hand R. Since the endoscope 100 and the operation device 300 are separated, the operator S can operate the endoscope 100 and the operation device 300 independently without them being influenced by each other.

### Endoscope 100

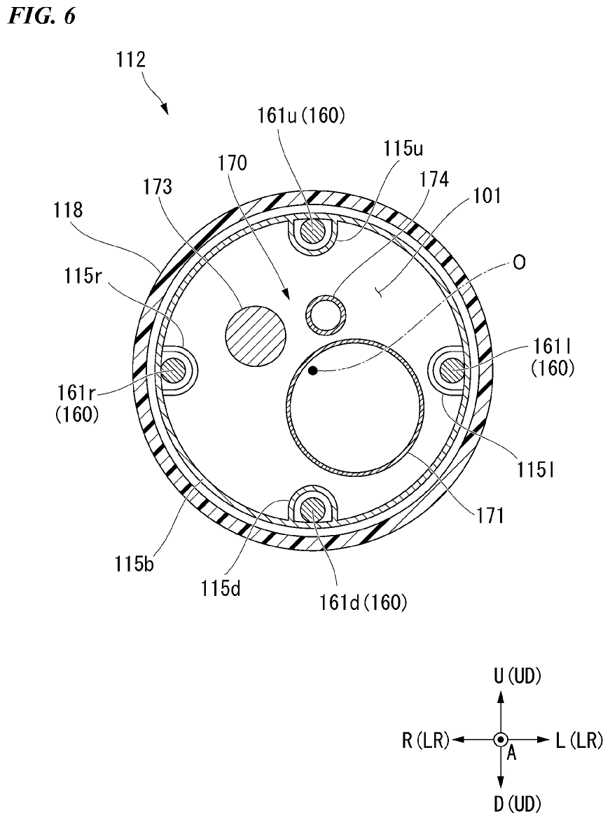

As shown in FIG. 1, the endoscope 100 includes an insertion portion 110, a connecting portion 120, an extracorporeal flexible portion 140, an attachment and detachment portion 150, a wire 160 (refer to FIG. 6), and a built-in object 170 (refer to FIG. 6). The insertion portion 110, the connecting portion 120, the extracorporeal flexible portion 140, and the attachment and detachment portion 150 are connected in this order from the distal end side.

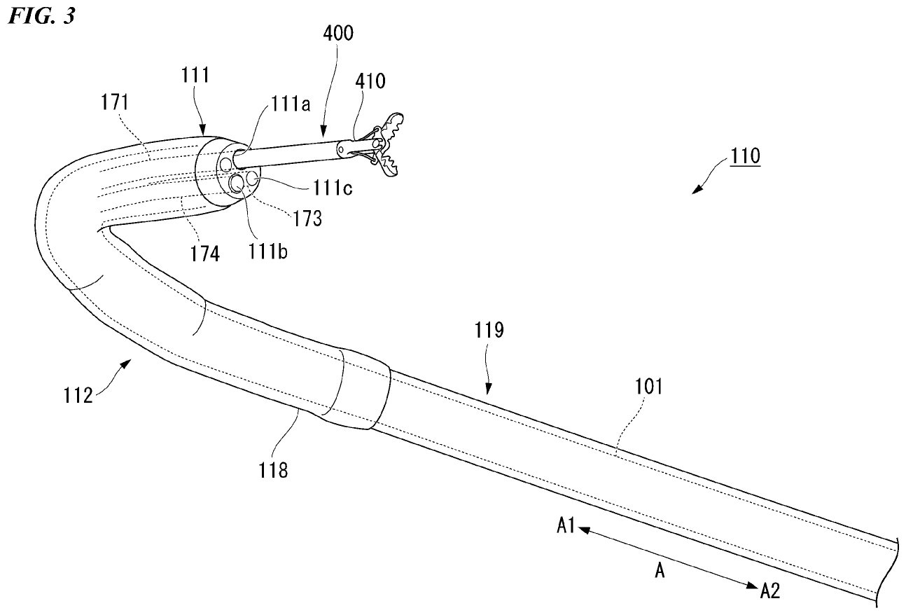

FIG. 3 is a view showing the insertion portion 110 of the endoscope 100.

The internal path 101 that extends in a longitudinal direction A of the endoscope 100 from a distal end of the insertion portion 110 to a proximal end of the attachment and detachment portion 150 is formed inside the endoscope 100. The wire 160 and the built-in object 170 are inserted into the internal path 101.

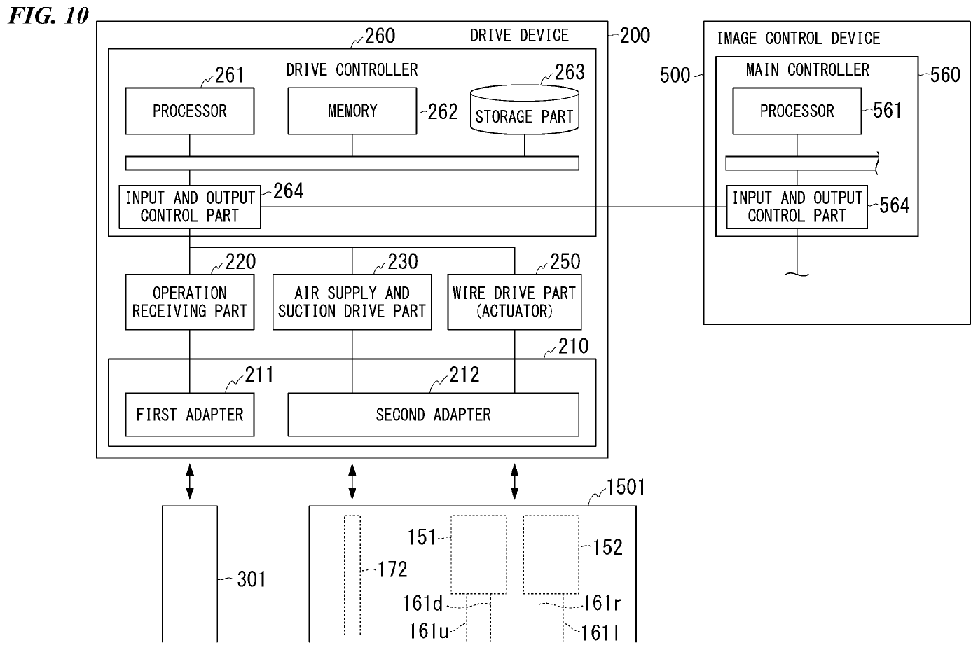

The built-in object 170 includes a channel tube 171, an air supply and suction tube 172 (refer to FIG. 10), an imaging cable 173, and a light guide 174.

### Insertion Portion 110

The insertion portion 110 is an elongated member that can be inserted into the lumen. The insertion portion 110 has a distal end portion 111, a bending portion 112, and an intracorporeal flexible portion 119. The distal end portion 111, the bending portion 112, and the intracorporeal flexible portion 119 are connected in order from the distal end side.

As shown in FIG. 3, the distal end portion 111 includes an opening portion 111a, an illumination part 111b, and an imaging part 111c. The opening portion 111a is an opening that communicates with the channel tube 171. As shown in FIG. 3, a treatment portion 410, such as a gripping forceps, provided at the distal end of the treatment tool 400 inserted through the channel tube 171 protrudes and retracts from the opening portion 111a.

The illumination part 111b is connected to the light guide 174 that guides illumination light, and emits illumination light that illuminates an imaging target. The imaging part 111c includes an imaging device such as a CMOS, and images the imaging target. An imaging signal is sent to the image control device 500 via the imaging cable 173.

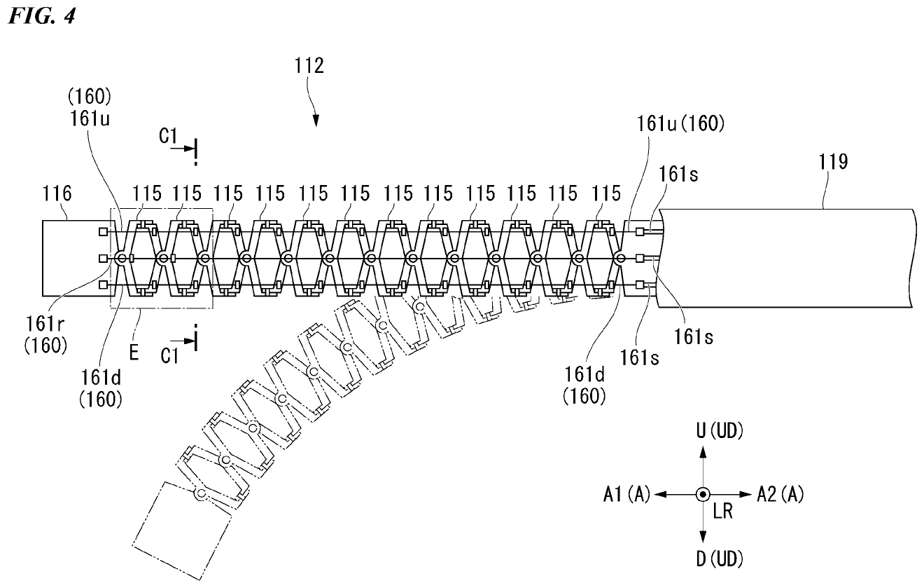

FIG. 4 is a view showing a part of the bending portion 112 as a cross-sectional view.

The bending portion 112 includes a plurality of joint rings (also referred to as bending pieces) 115, a distal end portion 116 connected to the distal ends of the plurality of joint rings 115, and an outer sheath 118 (refer to FIG. 3). The plurality of joint rings 115 and the distal end portion 116 are connected in the longitudinal direction A inside the outer sheath 118. The shape and number of the joint rings 115 that the bending portion 112 includes are not limited to the shape and number of the joint rings 115 shown in FIG. 4.

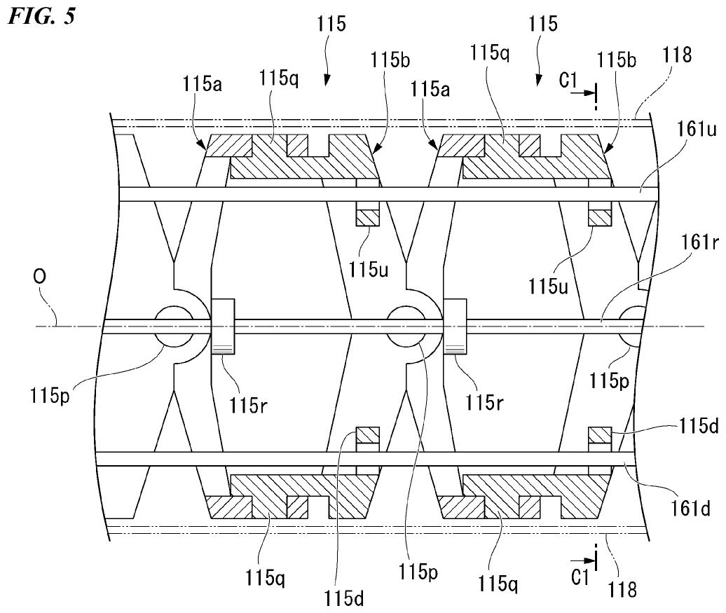

FIG. 5 is an enlarged view of the joint ring 115 in a region E shown in FIG. 4.

Each of the joint rings 115 is a short cylindrical member made of a metal. The plurality of joint rings 115 are connected to each other so that internal spaces of the adjacent joint rings 115 are continuous spaces.

Each of the joint rings 115 includes a first joint ring 115a on the distal end side and a second joint ring 115b on the proximal end side. The first joint ring 115a and the second joint ring 115b are connected to be rotatable in an up-down direction (also referred to as a “UD direction”) perpendicular to the longitudinal direction A by a first rotation pin 115p.

In the adjacent joint rings 115, the second joint ring 115b of the joint ring 115 on the distal end side and the first joint ring 115a of the joint ring 115 on the proximal end side are connected to be rotatable in a left-right direction (also referred to as an “LR direction”) perpendicular to the longitudinal direction A and the UD direction by a second rotation pin 115q.

The first joint ring 115a and the second joint ring 115b are alternately connected by the first rotation pin 115p and the second rotation pin 115q, and the bending portion 112 can be bent in a desired direction.

FIG. 6 is a cross-sectional view of the bending portion 112 taken along line C1-C1 in FIGS. 4 and 5.

An upper wire guide 115u and a lower wire guide 115d are formed on the inner circumferential surface of the second joint ring 115b. The upper wire guide 115u and the lower wire guide 115d are disposed on both sides of a central axis O in the UD direction with the central axis O in the longitudinal direction A interposed therebetween. A left wire guide 115l and a right wire guide 115r are formed on the inner circumferential surface of the first joint ring 115a. The left wire guide 115l and the right wire guide 115r are disposed on both sides of the central axis O in the LR direction with the central axis O in the longitudinal direction A interposed therebetween.

Through holes through which the wire 160 is inserted are formed in the longitudinal direction A in the upper wire guide 115u, the lower wire guide 115d, the left wire guide 115l, and the right wire guide 115r.

The wire 160 is a wire that bends the bending portion 112. The wire 160 extends through the internal path 101 to the attachment and detachment portion 150. As shown in FIGS. 4 and 6, the wire 160 includes an upper wire 161u, a lower wire 161d, a left wire 161l, a right wire 161r, and four wire sheaths 161s.

As shown in FIG. 4, the upper wire 161u, the lower wire 161d, the left wire 161l, and the right wire 161r are respectively inserted through the wire sheaths 161s. The distal end of the wire sheath 161s is mounted in the joint ring 115 at the proximal end of the bending portion 112. The wire sheaths 161s extend to the attachment and detachment portion 150.

The upper wire 161u and the lower wire 161d are wires that bend the bending portion 112 in the UD direction. The upper wire 161u passes through the upper wire guide 115u. The lower wire 161d passes through the lower wire guide 115d.

The distal ends of the upper wire 161u and the lower wire 161d are fixed to the distal end portion 116 at the distal end of the bending portion 112, as shown in FIG. 4. The distal ends of the upper wire 161u and the lower wire 161d fixed to the distal end portion 116 are disposed on both sides of the central axis O in the UD direction with the central axis O in the longitudinal direction A interposed therebetween.

The left wire 161l and the right wire 161r are wires that bend the bending portion 112 in the LR direction. The left wire 161l passes through the left wire guide 115l. The right wire 161r passes through the right wire guide 115r.

The distal ends of the left wire 161l and the right wire 161r are fixed to the distal end portion 116 of the bending portion 112, as shown in FIG. 4. The distal ends of the left wire 161l and the right wire 161r fixed to the distal end portion 116 are disposed on both sides of the central axis O in the LR direction with the central axis O in the longitudinal direction A interposed therebetween.

The bending portion 112 can be bent in a desired direction by pulling or relaxing each of the wires 160 (the upper wire 161u, the lower wire 161d, the left wire 161l, the right wire 161r).

As shown in FIG. 6, the wire 160, the channel tube 171, the imaging cable 173, and the light guide 174 are inserted through the internal path 101 formed inside the bending portion 112.

The intracorporeal flexible portion 119 is a long and flexible tubular member. The wire 160, the channel tube 171, the imaging cable 173, and the light guide 174 are inserted through the internal path 101 formed in the intracorporeal flexible portion 119.

### Connecting Portion 120

The connecting portion 120 is a member that connects the intracorporeal flexible portion 119 to the extracorporeal flexible portion 140 of the insertion portion 110, as shown in FIG. 1. The connecting portion 120 has the forceps port 126 that is an insertion port into which the treatment tool 400 is inserted.

### Extracorporeal Flexible Portion 140

The extracorporeal flexible portion 140 is a long tubular member. The wire 160, the imaging cable 173, the light guide 174, and the air supply and suction tube 172 (refer to FIG. 10) are inserted through the internal path 101 formed inside the extracorporeal flexible portion 140.

### Attachment and Detachment Portion 150

As shown in FIG. 1, the attachment and detachment portion 150 includes a first attachment and detachment portion 1501 that is mounted on the drive device 200 and a second attachment and detachment portion 1502 that is mounted on the image control device 500. The first attachment and detachment portion 1501 and the second attachment and detachment portion 1502 may be an integrated attachment and detachment portion.

The internal path 101 formed inside the extracorporeal flexible portion 140 branches into the first attachment and detachment portion 1501 and the second attachment and detachment portion 1502. The wire 160 and the air supply and suction tube 172 are inserted through the first attachment and detachment portion 1501. The imaging cable 173 and the light guide 174 are inserted through the second attachment and detachment portion 1502.

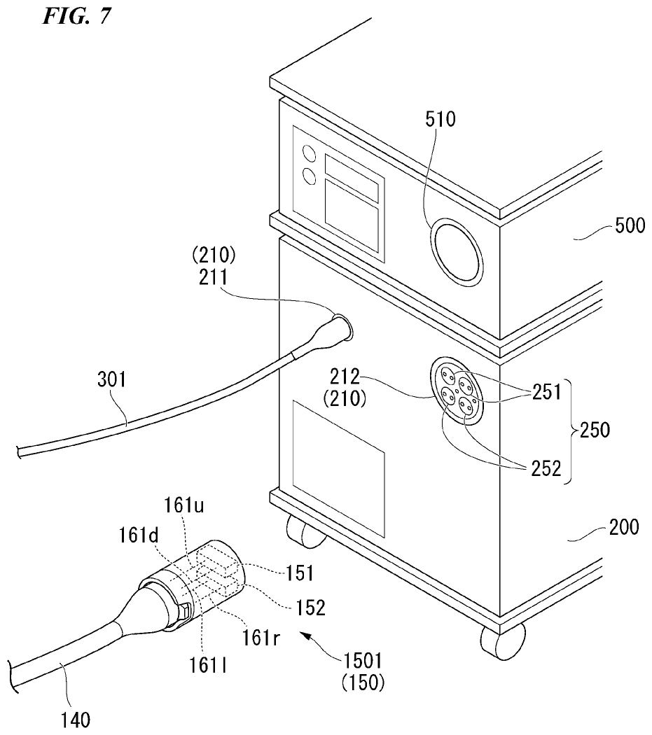

FIG. 7 is a view showing the first attachment and detachment portion 1501 before being mounted in the drive device 200.

The first attachment and detachment portion 1501 includes an up-down wire attachment and detachment portion 151 and a left-right wire attachment and detachment portion 152.

The up-down wire attachment and detachment portion 151 is a mechanism for detachably connecting the wires (the upper wire 161u and the lower wire 161d) that bend the bending portion 112 in the UD direction to the drive device 200.

The left-right wire attachment and detachment portion 152 is a mechanism for detachably connecting the wires (the left wire 161l and the right wire 161r) that bend the bending portion 112 in the LR direction to the drive device 200.

Since the left-right wire attachment and detachment portion 152 has the same structure as the up-down wire attachment and detachment portion 151, illustration and description thereof will be omitted.

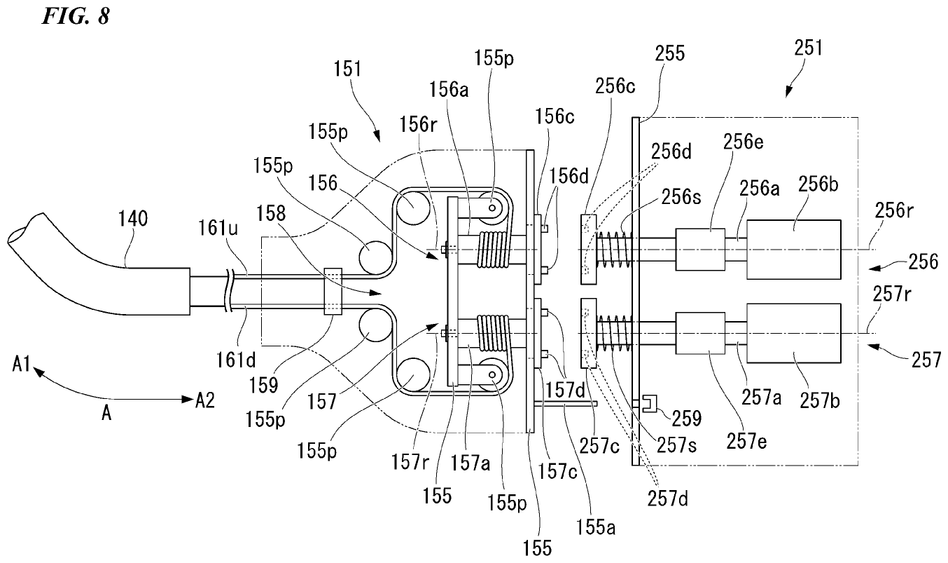

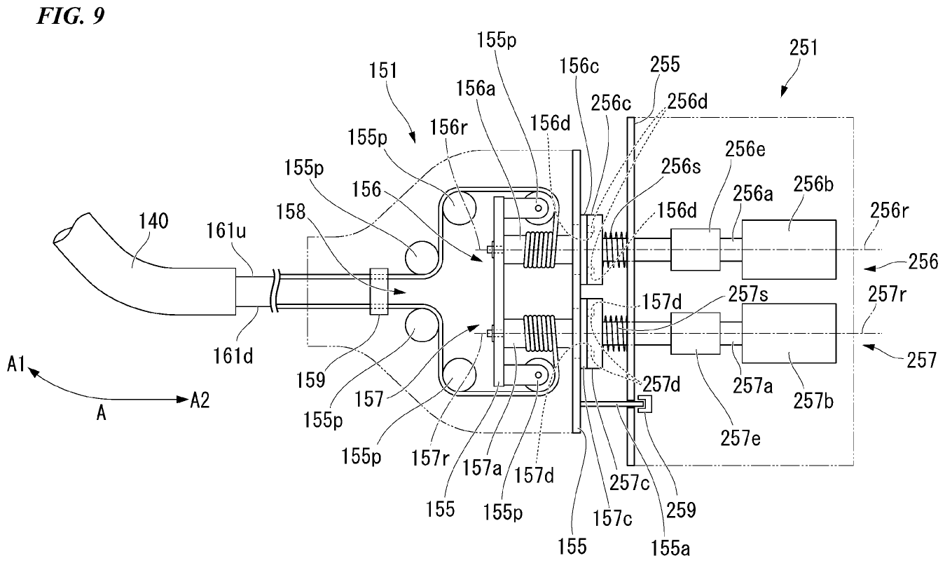

FIG. 8 is a view showing the up-down wire attachment and detachment portion 151 before being mounted in the drive device 200. FIG. 9 is a view showing the up-down wire attachment and detachment portion 151 mounted in the drive device 200. The up-down wire attachment and detachment portion 151 includes a support member 155, a first rotating drum 156, a second rotating drum 157, and a tension sensor 159.

The support member 155 supports the first rotating drum 156, the second rotating drum 157, and a connecting member 158. The support member 155 includes an attachment and detachment detection dog 155a exposed on the proximal end side of the up-down wire attachment and detachment portion 151, and a plurality of bend pulleys 155p.

The bend pulley 155p changes a transport direction of the upper wire 161u that is inserted through the extracorporeal flexible portion 140, and guides the upper wire 161u to the first rotating drum 156. Further, the bend pulley 155p changes a transport direction of the lower wire 161d that is inserted through the extracorporeal flexible portion 140, and guides the lower wire 161d to the second rotating drum 157.

The first rotating drum 156 is supported by the support member 155 to be rotatable around a first drum rotation axis 156r extending in the longitudinal direction A. The first rotating drum 156 includes a first winding pulley 156a and a first coupling portion 156c.

The first winding pulley 156a pulls or sends out the upper wire 161u by rotating around the first drum rotation axis 156r. As the first winding pulley 156a rotates clockwise when seen from the distal end side toward the proximal end side, the upper wire 161u is wound around the first winding pulley 156a and pulled. Conversely, as the first winding pulley 156a rotates counterclockwise, the upper wire 161u is sent out of the first winding pulley 156a. With this configuration, even when the upper wire 161u advances and retreats by a large amount, the pulled portion is stored compactly and does not take up much space.

The first coupling portion 156c is a disc member that rotates around the first drum rotation axis 156r. The first coupling portion 156c is fixed to the proximal end of the first winding pulley 156a, and rotates integrally with the first winding pulley 156a. The first coupling portion 156c is exposed on the proximal end side of the up-down wire attachment and detachment portion 151. Two first fitting convex portions 156d are formed on a surface of the first coupling portion 156c on the proximal end side. The two first fitting convex portions 156d are formed on both sides of the first drum rotation axis 156r with the first drum rotation axis 156r interposed therebetween.

The second rotating drum 157 is supported by the support member 155 to be rotatable around a second drum rotation axis 157r extending along the longitudinal direction A. The second rotating drum 157 includes a second winding pulley 157a and a second coupling portion 157c.

The second winding pulley 157a pulls or sends out the lower wire 161d by rotating around the second drum rotation axis 157r. As the second winding pulley 157a rotates counterclockwise when seen from the distal end side to the proximal end side, the lower wire 161d is wound around the second winding pulley 157a and pulled. Conversely, as the second winding pulley 157a rotates clockwise, the lower wire 161d is sent out of the second winding pulley 157a.

The second coupling portion 157c is a disc member that rotates around the second drum rotation axis 157r. The second coupling portion 157c is fixed to the proximal end of the second winding pulley 157a, and rotates integrally with the second winding pulley 157a. The second coupling portion 157c is exposed on the proximal end side of the up-down wire attachment and detachment portion 151. Two second fitting convex portions 157d are formed on a surface of the second coupling portion 157c on the proximal end side. The two second fitting convex portions 157d are formed on both sides of the second drum rotation axis 157r with the second drum rotation axis 157r interposed therebetween.

The tension sensor 159 detects a tension of each of the upper wire 161u and the lower wire 161d. Detection results of the tension sensor 159 are acquired by a drive controller 260.

### Drive Device 200

FIG. 10 is a functional block diagram of the drive device 200.

The drive device 200 includes an adapter 210, an operation receiving part 220, an air supply and suction drive part 230, a wire drive part (an actuator) 250, and the drive controller 260.

As shown in FIG. 7, the adapter 210 includes a first adapter 211 and a second adapter 212. The first adapter 211 is an adapter to which the operation cable 301 is detachably connected. The second adapter 212 is an adapter to which the first attachment and detachment portion 1501 of the endoscope 100 is detachably connected.

The operation receiving part 220 receives an operation input from the operation device 300 via the operation cable 301. When the operation device 300 and the drive device 200 communicate by wireless communication instead of wired communication, the operation receiving part 220 includes a known wireless receiving module.

The air supply and suction drive part 230 is connected to the air supply and suction tube 172 inserted into the internal path 101 of the endoscope 100. The air supply and suction drive part 230 includes a pump and the like, and supplies air to the air supply and suction tube 172. Further, the air supply and suction drive part 230 suctions air from the air supply and suction tube 172.

The wire drive part (the actuator) 250 is coupled with the up-down wire attachment and detachment portion 151 and the left and right wire attachment and detachment portion 152 to drive the wire 160.

As shown in FIG. 7, the wire drive part 250 includes an up-down wire drive part (a first actuator) 251 and a left-right wire drive part (a second actuator) 252.

The up-down wire drive part 251 is a mechanism that is coupled with the up-down wire attachment and detachment portion 151 and drives the wires (the upper wire 161u and the lower wire 161d) that bend the bending portion 112 in the UD direction.

The left-right wire drive part 252 is a mechanism that is coupled with the left-right wire attachment and detachment portion 152 and drives the wires (the left wire 161l and the right wire 161r) that bend the bending portion 112 in the LR direction.

Since the left-right wire drive part 252 has the same structure as the up-down wire drive part 251, illustration and description thereof will be omitted.

As shown in FIG. 8, the up-down wire drive part 251 includes a support member 255, an upper wire drive part 256, a lower wire drive part 257, and an attachment and detachment sensor 259.

The upper wire drive part 256 is coupled with the first rotating drum 156 of the up-down wire attachment and detachment portion 151 to drive the upper wire 161u. The upper wire drive part 256 includes a first shaft 256a, a first motor part 256b, a first coupled portion 256c, a first torque sensor 256e, and a first elastic member 256s.

The first shaft 256a is supported by the support member 255 so as to be rotatable around a first shaft rotation axis 256r and to be advanceable and retractable in the longitudinal direction A. When the first attachment and detachment portion 1501 of the endoscope 100 is mounted in the drive device 200, the first shaft rotation axis 256r coincides with the first drum rotation axis 156r.

The first motor part 256b includes a first motor such as a DC motor, a first motor driver that drives the first motor, and a first motor encoder. The first motor rotates the first shaft 256a around the first shaft rotation axis 256r. The first motor driver is controlled by the drive controller 260.

The first coupled portion 256c is a disc member that rotates around the first shaft rotation axis 256r. The first coupled portion 256c is fixed to the distal end of the first shaft 256a, and rotates integrally with the first shaft 256a. As shown in FIG. 8, the first coupled portion 256c is exposed on the distal end side of the up-down wire drive part 251. Two first fitting concave portions 256d are formed in a surface of the first coupled portion 256c on the distal end side. The two first fitting concave portions 256d are formed on both sides of the first shaft rotation axis 256r with the first shaft rotation axis 256r interposed therebetween.

As shown in FIG. 9, the first fitting convex portion 156d and the first fitting concave portion 256d are fitted to each other, and the first coupling portion 156c and the first coupled portion 256c are coupled. As a result, rotation of the first shaft 256a by the first motor part 256b is transmitted to the first rotating drum 156. As the first shaft 256a rotates clockwise when seen from the distal end side to the proximal end side, the upper wire 161u is pulled. Conversely, when the first shaft 256a rotates counterclockwise, the upper wire 161u is sent out.

The first torque sensor 256e detects rotational torque of the first shaft 256a around the first shaft rotation axis 256r. The detection result of the first torque sensor 256e is acquired by the drive controller 260.

The first elastic member 256s is, for example, a compression spring, and has a distal end in contact with the first coupled portion 256c and a proximal end in contact with the support member 255. The first elastic member 256s biases the first coupled portion 256c toward the distal end side A1. As shown in FIG. 9, when the first coupling portion 156c is mounted, the first coupled portion 256c moves toward the proximal end side A2 together with the first shaft 256a.

The lower wire drive part 257 is coupled with the second rotating drum 157 of the up-down wire attachment and detachment portion 151 to drive the lower wire 161d. The lower wire drive part 257 includes a second shaft 257a, a second motor part 257b, a second coupled portion 257c, a second torque sensor 257e, and a second elastic member 257s.

The second shaft 257a is supported by the support member 255 so as to be rotatable around the second shaft rotation axis 257r and to be advanceable and retractable in the longitudinal direction A. When the first attachment and detachment portion 1501 of the endoscope 100 is mounted in the drive device 200, the second shaft rotation axis 257r coincides with the second drum rotation axis 157r.

The second motor part 257b includes a second motor such as a DC motor, a second motor driver that drives the second motor, and a second motor encoder. The second motor rotates the second shaft 257a around the second shaft rotation axis 257r. The second motor driver is controlled by the drive controller 260.

The second coupled portion 257c is a disc member that rotates around the second shaft rotation axis 257r. The second coupled portion 257c is fixed to the distal end of the second shaft 257a, and rotates integrally with the second shaft 257a. As shown in FIG. 8, the second coupled portion 257c is exposed on the distal end side of the up-down wire drive part 251. Two second fitting concave portions 257d are formed in a surface of the second coupled portion 257c on the distal end side. The two second fitting concave portions 257d are formed on both sides of the second shaft rotation axis 257r with the second shaft rotation axis 257r interposed therebetween.

As shown in FIG. 9, the second fitting convex portion 157d and the second fitting concave portion 257d are fitted to each other, and the second coupling portion 157c and the second coupled portion 257c are coupled. As a result, rotation of the second shaft 257a by the second motor part 257b is transmitted to the second rotating drum 157. As the second shaft 257a rotates counterclockwise when seen from the distal end side to the proximal end side, the lower wire 161d is pulled. Conversely, when the second shaft 257a rotates clockwise, the lower wire 161d is sent out.

The second torque sensor 257e detects rotational torque of the second shaft 257a around the second shaft rotation axis 257r. The detection result of the second torque sensor 257e is acquired by the drive controller 260.

The second elastic member 257s is, for example, a compression spring, and has a distal end in contact with the second coupled portion 257c and a proximal end in contact with the support member 255. The second elastic member 257s biases the second coupled portion 257c toward the distal end side A1. As shown in FIG. 9, when the second coupling portion 157c is mounted, the second coupled portion 257c moves toward the proximal end side A2 together with the second shaft 257a.

As shown in FIG. 9, the attachment and detachment sensor 259 detects attachment and detachment of the up-down wire attachment and detachment portion 151 to/from the up-down wire drive part 251 by detecting engagement and disengagement with the attachment and detachment detection dog 155a. The detection result of the attachment and detachment sensor 259 is acquired by the drive controller 260.

With the above mechanism, when the up-down wire attachment and detachment portion 151 is mounted on the up-down wire drive part 251, the upper wire drive part 256 can independently drive the upper wire 161u, and the lower wire drive part 257 can independently drive the lower wire 161d. Therefore, even when a distance from the bending portion 112 of the endoscope 100 to the drive device 200 is longer than that of a conventional flexible endoscope, a bending operation of the bending portion 112 can be controlled with high precision.

The drive controller 260 controls the entire drive device 200. The drive controller 260 acquires an operation input received by the operation receiving part 220. The drive controller 260 controls the air supply and suction drive part 230 and the wire drive part 250 on the basis of the acquired operation input.

The drive controller 260 is a computer capable of executing programs that includes a processor 261, a memory 262, a storage part 263 that can store programs and data, and an input and output control part 264. Functions of the drive controller 260 are realized by a processor executing a program. At least some of the functions of drive controller 260 may be realized by a dedicated logic circuit.

The drive controller 260 preferably has high calculation performance in order to control the plurality of motors that drive the plurality of wires 160 with high precision.

The drive controller 260 may further include configurations other than the processor 261, the memory 262, the storage part 263, and the input and output control part 264. For example, the drive controller 260 may further include an image calculation part that performs part or all of image processing and image recognition processing. The drive controller 260 can perform specific image processing and image recognition processing at high speed by further including the image calculation part. The image calculation part may be mounted in a separate hardware device connected via a communication line.

### Operation Device 300

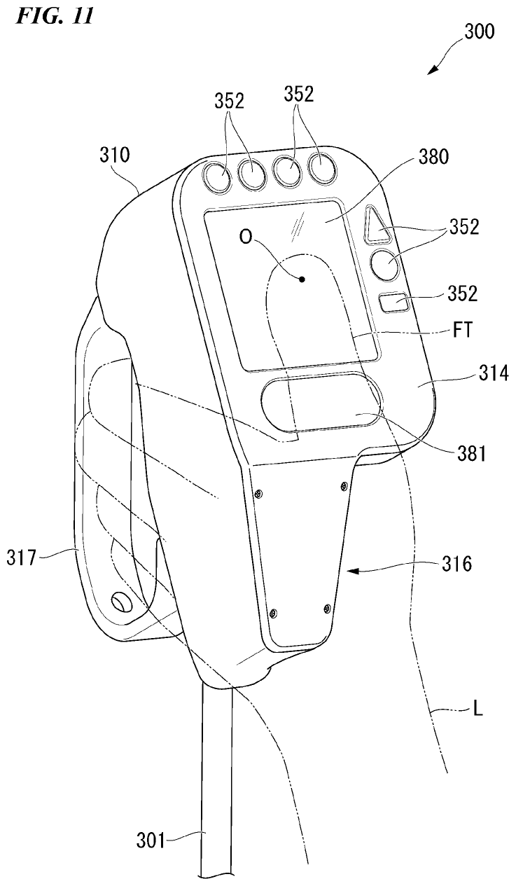

FIG. 11 is a perspective view of the operation device 300.

The operation device 300 is a device into which an operation for driving the endoscope 100 is input. The input operation input is transmitted to the drive device 200 via the operation cable 301. The operation device 300 may be able to communicate with the drive device 200 by wireless communication instead of wired communication.

The operation device 300 includes an operation part main body 310, an air supply button, a suction button, various buttons 352, a touch pad 380, and a touch sensor 381.

The operation part main body 310 is formed into a substantially rod shape that can be held by the operator S with the left hand L. The operation part main body 310 includes a touch pad support portion 314 provided above, a grip portion 316 provided below, and a handle 317 provided at the rear. As shown in FIG. 11, the operator S can operate the touch pad 380 with the thumb FT of the left hand L while gripping the grip portion 316 with the left hand L.

The touch pad 380 is a touch-sensitive interface through which a bending operation for the bending portion 112 and the like are input. The touch pad 380 may be a touch panel.

### Image Control Device 500

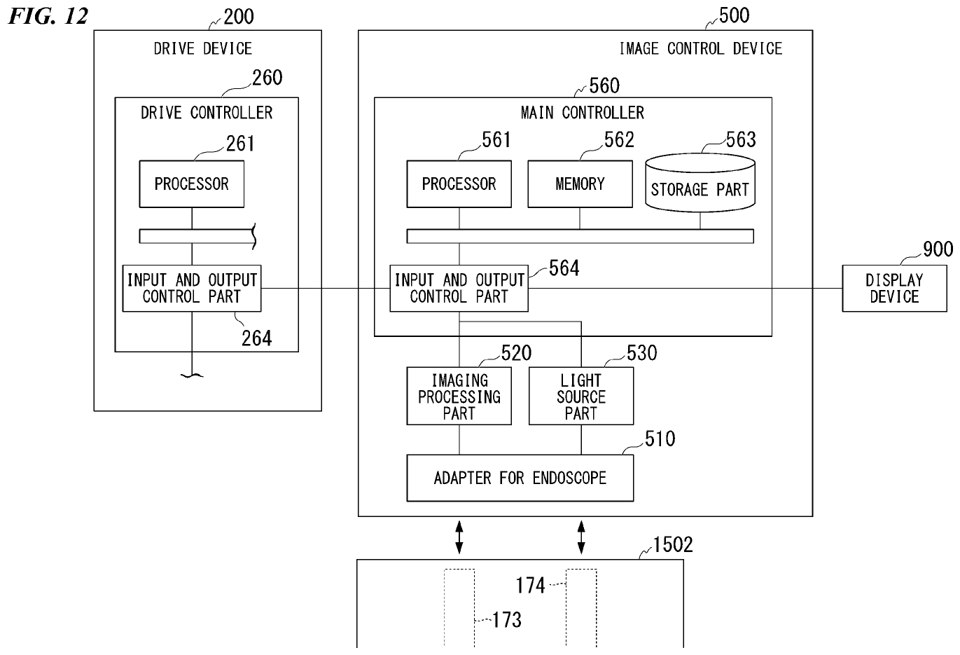

FIG. 12 is a functional block diagram of the image control device 500.

The image control device 500 controls the electric endoscope system 1000. The image control device 500 includes a third adapter 510, an imaging processing part 520, a light source part 530, and a main controller 560.

The third adapter 510 is an adapter to which the second attachment and detachment portion 1502 of the endoscope 100 is detachably connected.

The imaging processing part 520 converts an imaging signal acquired from the imaging part 111c of the distal end portion 111 via the imaging cable 173 into a captured image.

The light source part 530 generates illumination light that is radiated onto an imaging target. The illumination light generated by the light source part 530 is guided to the illumination part 111b of the distal end portion 111 via the light guide 174.

The main controller 560 is a computer capable of executing programs that includes a processor 561, a memory 562, a storage part 563 that can store programs and data, and an input and output control part 564. Functions of the main controller 560 are realized by the processor 561 executing a program. At least some of the functions of main controller 560 may be realized by a dedicated logic circuit.

The main controller 560 includes the processor 561, the memory 562 into which a program can be read, the storage part 563, and the input and output control part 564.

The storage part 563 is a nonvolatile recording medium that stores the above-described programs and necessary data. The storage part 563 is configured of, for example, a ROM or a hard disk. The program recorded in storage part 563 is read into the memory 562 and is executed by the processor 561.

The input and output control part 564 is connected to the imaging processing part 520, the light source part 530, the drive device 200, the display device 900, an input device (not shown), and a network device (not shown). The input and output control part 564 transmits and receives data and control signals to and from connected devices under the control of the processor 561.

The main controller 560 can perform image processing on the captured image acquired by the imaging processing part 520. The main controller 560 can generate GUI images and CG images for the purpose of providing information to the operator S. The main controller 560 can display the captured images, the GUI images, and the CG images on the display device 900.

The main controller 560 is not limited to an integrated hardware device. For example, the main controller 560 may be configured by separating a part thereof as a separate hardware device and then connecting the separated hardware device via a communication line. For example, the main controller 560 may be a cloud system that connects the separate storage part 563 via a communication line.

The main controller 560 may further include configurations other than the processor 561, the memory 562, the storage part 563, and the input and output control part 564 shown in FIG. 12. For example, the main controller 560 may further include an image calculation part that performs part or all of the image processing and image recognition processing that the processor 561 has performed. The main controller 560 can perform specific image processing and image recognition processing at high speed by further including an image calculation part. The image calculation part may be mounted in a separate hardware device connected via a communication line.

### Operation of Electric Endoscope System 1000

Next, the operation of the electric endoscope system 1000 of this embodiment will be described. Specifically, a technique for observing and treating an affected area formed on the wall of the large intestine using the electric endoscope system 1000 will be described.

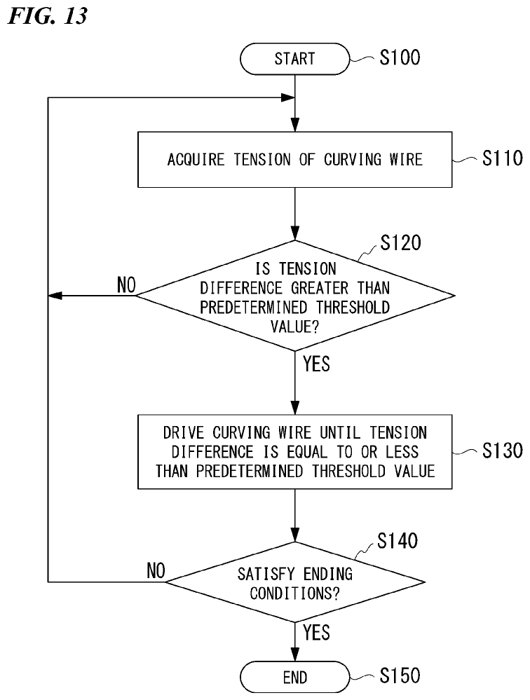

Hereinafter, the description will be given along a control flowchart of the drive controller 260 of the control device 600 shown in FIG. 13. When the control device 600 is activated, the drive controller 260 starts angle-free control (also called tension control) after performing initialization (Step S100). Next, the drive controller 260 (mainly the processor 261) performs Step S110.

When the control device 600 is activated, the angle-free control may be disabled. The angle-free control described below may be enabled only when a predetermined operation input from the operator S is made to a control button for the angle-free control assigned to the various buttons 352 of the operation device 300.

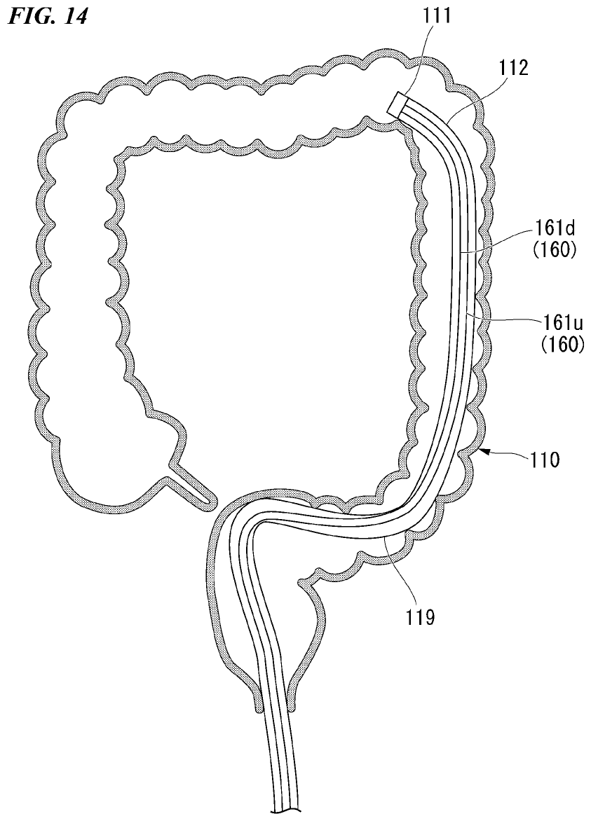

FIG. 14 is a view showing the insertion portion 110 inserted into the large intestine.

The operator S inserts the insertion portion 110 of the endoscope 100 into the large intestine of the patient P through the anus. The operator S moves the insertion portion 110 while observing the captured image displayed on the display device 900, operating the intracorporeal flexible portion 119 with the right hand R, and brings the distal end portion 111 closer to the affected area. Further, the operator S operates the operation device 300 with the left hand L to input a bending operation to the bending portion 112.

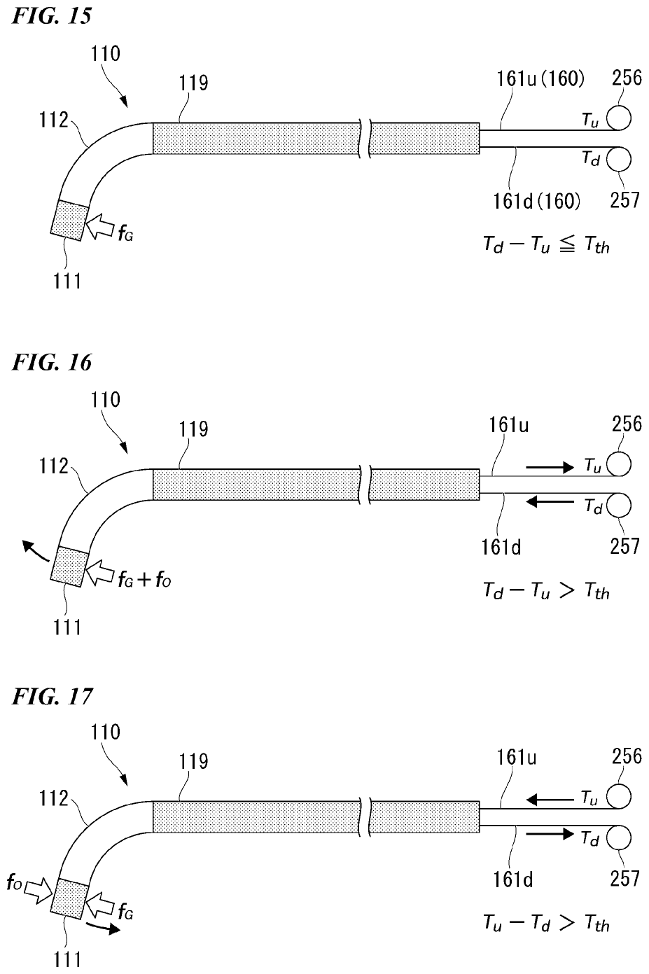

FIG. 15 is a view showing the bending portion 112.

The drive controller 260 performs bending control on the bending portion 112 on the basis of the received bending operation, and controls the wire drive part (the actuator) 250 to bend the wire 160. A restoring force f_{G} which tends to return the bending portion 112 made of rubber or the like forming the outer sheath 118 to a straight state acts on the bending portion 112.

### Step S110

The drive controller 260 acquires tension of the wire 160 from the tension sensor 159 in Step S110. The drive controller 260 may estimate the tension of the wire 160 from torque acquired from the torque sensors (the first torque sensor 256e, the second torque sensor 257e) of the drive device 200 and motor current values of the motor parts (the first motor part 256b, the second motor part 257b). The drive controller 260 then performs Step S120.

### Step S120

In Step S110, the drive controller 260 compares a tension difference (an absolute value) between the pair of wires 160 (the upper wire 161u and the lower wire 161d) that bends the bending portion 112 in the up-down direction (the UD direction) with a predetermined threshold value Tth.

FIG. 16 is a view showing an example of the bending portion 112 in which the tension difference equal to or larger than the predetermined threshold value has occurred.

In the example shown in FIG. 16, the tension Td of the lower wire 161d increases by applying an external force f_{O} in a direction in which the bending portion 112 is returned to the straight state, and the tension difference between the tension Tu of the upper wire 161u and the tension Td of the lower wire 161d becomes larger than the predetermined threshold value Tth.

FIG. 17 is a view showing another example of the bending portion 112 in which the tension difference equal to or larger than the predetermined threshold value has occurred.

In the example shown in FIG. 17, the tension Tu of the upper wire 161u increases by applying the external force f_{O} in a direction in which the bending portion 112 is further bent, and the tension difference between the tension Tu of the upper wire 161u and the tension Td of the lower wire 161d becomes larger than the predetermined threshold value Tth.

When the tension difference is equal to or less than the predetermined threshold value Tth, the drive controller 260 next performs Step S110. When the tension difference is greater than the predetermined threshold value Tth, the drive controller 260 next performs Step S130.

### Step S130

In Step S130, the drive controller 260 drives the wire 160 until the tension difference between the pair of wires 160 becomes equal to or less than the predetermined threshold value Tth (first tension control). The bending portion 112 is driven by the wire drive part (the actuator) 250 and bends in a direction to which the external force f_{O} is applied.

In the angle-free control, the drive controller 260 drives the wire 160 by, for example, substantially matching an amount of sending of one of the pair of wires 160 having a higher tension with an amount of pulling of the other wire 160 (push-pull control). In the example shown in FIG. 16, the drive controller 260 pulls the upper wire 161u and sends out the lower wire 161d. In the example shown in FIG. 17, the drive controller 260 pulls the lower wire 161d and sends out the upper wire 161u.

In the angle-free control, the drive controller 260 drives the wire 160 so that, for example, one of the pair of wires 160 having a higher tension is sent out, and the tension of the other wire 160 is kept constant (antagonistic control). In the example shown in FIG. 16, the drive controller 260 sends out the lower wire 161d and keeps the tension of the upper wire 161u constant. In the example shown in FIG. 17, the drive controller 260 sends out the upper wire 161u and keeps the tension of the lower wire 161d constant.

In the angle-free control, the drive controller 260 drives the wire drive part (the actuator) 250 so that the bending portion 112 bends at a constant speed (uniform speed control), for example. Thus, since the bending portion 112 bends at a constant speed, the operator S can easily recognize a bending motion of the bending portion 112.

In the angle-free control, the drive controller 260 drives the wire drive part (the actuator) 250 so that a speed at which the bending portion 112 operates changes on the basis of the tension difference between the pair of wires 160 (speed change control). The drive controller 260 increases the speed at which the bending portion 112 operates as the tension difference between the pair of wires 160 increases. As the tension difference between the pair of wires 160 becomes smaller, the drive controller 260 slows down the speed at which the bending portion 112 operates. Thus, the operation of the insertion portion 110 that is electrically driven approaches the same operation as the insertion portion of an existing endoscope that is manually operated.

### Step S140

In Step S140, the drive controller 260 determines whether the angle-free control will be ended. When the drive controller 260 determines that the angle-free control is not to be ended, it performs Step S110. When the drive controller 260 determines to end the angle-free control, it performs Step S150 and ends the angle-free control.

When the external force f_{O} is applied and the tension difference between the pair of wires 160 is greater than the predetermined threshold value Tth, the bending portion 112 bends in the direction to which the external force f_{O} is applied. Therefore, for example, when the operator S moves the insertion portion 110 and the distal end portion 111 of the insertion portion 110 comes into contact with the wall of the lumen such as the large intestine, since the bending portion 112 bends in the direction to which the external force f_{O} is applied, it is difficult to apply an excessive force to the lumen such as the large intestine. Further, when the operator S removes the insertion portion 110 from the large intestine and the insertion portion 110 comes into contact with a bent portion of the large intestine, since the bending portion 112 bends in the direction to which the external force f_{O} is applied, and the bending portion 112 bends to match the shape of the bent portion of the lumen such as the large intestine, the operator S can easily remove the insertion portion 110.

The drive controller 260 performs the angle-free control by driving the wire drive part (the actuator) 250 in the same manner as the bending control for the bending portion 112 based on the bending operation for the bending portion 112 input from the operator S to the operation device 300. Therefore, the electric endoscope system 1000 does not require any additional mechanism or the like to perform the angle-free control. Further, in the angle-free control, since the drive controller 260 drives the bending portion 112 without loosening the wire 160 as in the bending control, the angle-free control and the bending control can be seamlessly switched.

The drive controller 260 may also perform the angle-free control when the bending control on the bending portion 112 is performed on the basis of the bending operation for the bending portion 112 input by the operator S. In the case in which the bending portion 112 is bent on the basis of the bending operation input by the operator S, when the tension difference between the pair of wires 160 becomes larger than the predetermined threshold value Tth due to the bending portion 112 comes into contact with the wall of the lumen such as the large intestine and the external force f_{O} being applied thereto, the drive controller 260 drives the wires 160 so that the tension difference between the pair of wires 160 is equal to or less than the predetermined threshold value Tth. That is, the drive controller 260 can drive the pair of wires 160 only within a range in which the tension difference between the pair of wires 160 is equal to or less than the predetermined threshold value Tth (limiter function). As a result, even when the bending portion 112 is bent on the basis of the bending operation input by the operator S, it does not bend further against the predetermined external force f_{O}, and thus it is difficult to apply an excessive force to the lumen such as the large intestine.

In the angle-free control, the drive controller 260 may change the predetermined threshold value Tth on the basis of the presence or absence of the bending operation on the operation device 300. For example, when the operator S inputs the bending operation, the drive controller 260 increases the predetermined threshold value Tth compared to when the operator S does not input the bending operation. Thus, the drive controller 260 can adjust a lower limit value of the external force f_{O} at which the limiter function due to the angle-free control operates.

The drive controller 260 may set the predetermined threshold value Tth small so that the tension difference between the pair of wires 160 becomes larger than the predetermined threshold value Tth when the restoring force f_{G} shown in FIG. 15 acts thereon. When the restoring force f_{G} is equal to or larger than a predetermined value, the drive controller 260 drives the wire drive part (the actuator) 250 so that the bending portion 112 is returned to the straight state. Thus, the operation of the insertion portion 110 that is electrically driven approaches the same operation as the insertion portion of an existing endoscope that is manually operated.

The drive controller 260 also performs the same angle-free control (second tension control) on the pair of wires 160 (the left wire 161l and the right wire 161r) that bend the bending portion 112 in the left-right direction (the LR direction). An initialization operation for the left wire 161l and the right wire 161r may be performed simultaneously with the angle-free control for the upper wire 161u and the lower wire 161d, or may be performed separately.

The angle-free control for the pair of wires 160 (the pair of wires 160 bent in the up-down direction, the pair of wires 160 bent in the left-right direction) may be set and changeable by the operator S. For example, the angle-free control for one or both of the pair of wires 160 may be enabled on the basis of a predetermined operation input from the operator S to a control button of the angle-free control assigned to the various buttons 352 of the operation device 300. The operator S can appropriately set the enablement of the angle-free control for the pair of wires 160 according to the type of treatment for the affected area.

Further, the predetermined threshold value Tth may be changed on the basis of a predetermined operation input from the operator S to the control button for the angle-free control assigned to the various buttons 352 of the operation device 300. The operator S can appropriately set the predetermined threshold value Tth according to the type of treatment for the affected area.

The above angle-free control may be performed by the main controller 560 (mainly the processor 561) controlling the wire drive part (the actuator) 250.

According to the electric endoscope system 1000 according to the present embodiment, it is possible to appropriately handle bending control (active control) in which the bending portion is actively driven by electric power, and angle-free control (passive control) in which the bending portion is passively driven when an external force is applied. The electric endoscope system 1000 can seamlessly switch between the bending control (the active control) and the angle-free control (the passive control). Furthermore, the electric endoscope system 1000 can also perform the bending control (the active control) and the angle-free control (the passive control) at the same time.

Although the first embodiment of the present invention has been described above in detail with reference to the drawings, the specific configuration is not limited to this embodiment, and design changes may be made within the scope of the gist of the present invention. Moreover, the components shown in the above-described embodiment and modified example can be configured by appropriately combining them.

### Second Embodiment

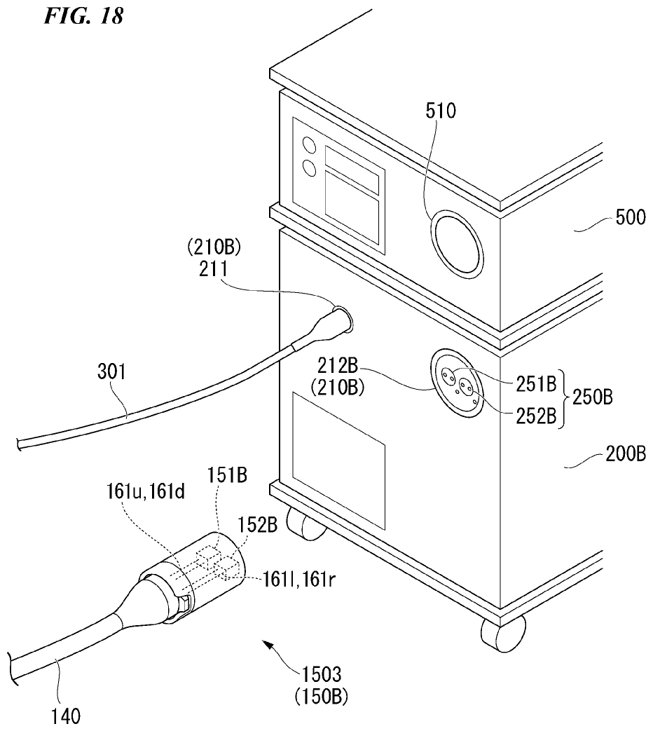

An electric endoscope system 1000B according to a second embodiment of the present invention will be described with reference to FIGS. 18 to 20. In the following description, components that are common to those already described will be designated by the same reference numerals, and redundant description will be omitted.

### Electric Endoscope System 1000B

As shown in FIG. 1, the electric endoscope system 1000B includes an endoscope 100B, a drive device 200B, an operation device 300, a treatment tool 400, an image control device 500, and a display device 900. The drive device 200B and the image control device 500 constitute a control device 600B that controls the electric endoscope system 1000B.

### Endoscope 100B

The endoscope 100B includes an insertion portion 110, a connecting portion 120, an extracorporeal flexible portion 140, an attachment and detachment portion 150B, a wire 160, and a built-in object 170.

### Attachment and Detachment Portion 150B

FIG. 18 is a view showing a first attachment and detachment portion 1503 before being mounted in the drive device 200B.

The attachment and detachment portion 150B includes the first attachment and detachment portion 1503 that is mounted in the drive device 200B, and a second attachment and detachment portion 1502 that is mounted in the image control device 500. The first attachment and detachment portion 1503 includes an up-down wire attachment and detachment portion 151B and a left-right wire attachment and detachment portion 152B.

The up-down wire attachment and detachment portion 151B is a mechanism for detachably connecting the wires (the upper wire 161u and the lower wire 161d) that bend the bending portion 112 in the UD direction to the drive device 200B.

The left-right wire attachment and detachment portion 152B is a mechanism for detachably connecting the wires (the left wire 161l and the right wire 161r) that bend the bending portion 112 in the LR direction to the drive device 200B.

The left-right wire attachment and detachment portion 152B has the same structure as the up-down wire attachment and detachment portion 151B, and thus illustration and description thereof will be omitted.

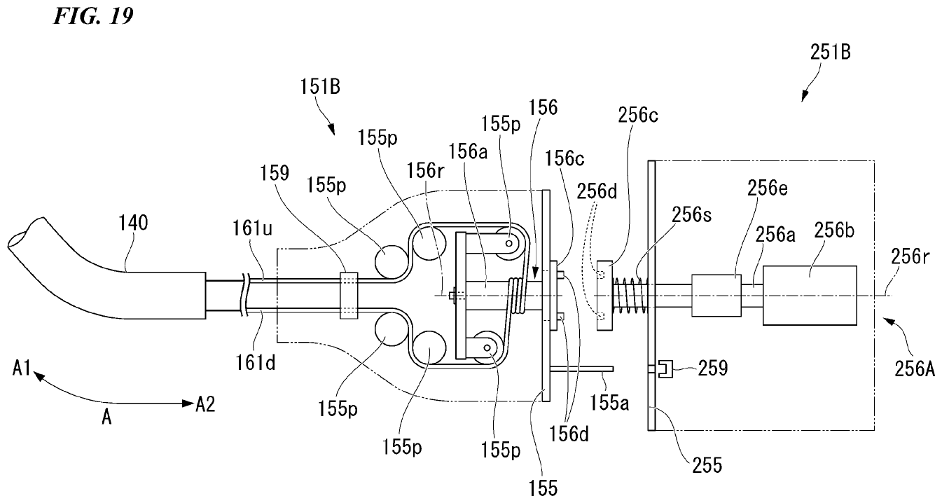

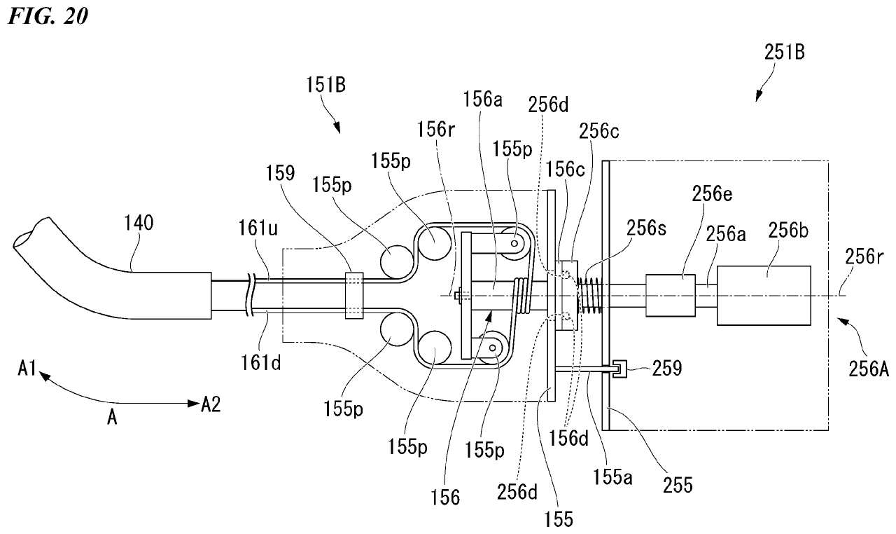

FIG. 19 is a view showing the up-down wire attachment and detachment portion 151B before being mounted in the drive device 200B. FIG. 20 is a view showing the up-down wire attachment and detachment portion 151B mounted in the drive device 200B. The up-down wire attachment and detachment portion 151B includes a support member 155, a rotating drum 156, and a tension sensor 159.

### Drive Device 200B

The drive device 200B includes an adapter 210B, an operation receiving part 220, an air supply and suction drive part 230, a wire drive part 250B, and a drive controller 260B.

As shown in FIG. 18, the adapter 210B includes a first adapter 211 and a second adapter 212B. The first adapter 211 is an adapter to which the operation cable 301 is detachably connected. The second adapter 212B is an adapter to which the first attachment and detachment portion 1503 of the endoscope 100 is detachably connected.

The wire drive part 250B is coupled with the up-down wire attachment and detachment portion 151B and the left-right wire attachment and detachment portion 152B to drive the wire 160.

As shown in FIG. 18, the wire drive part 250B includes an up-down wire drive part 251B and a left-right wire drive part 252B.

The up-down wire drive part 251B is a mechanism that is coupled with the up-down wire attachment and detachment portion 151B and drives the wires (the upper wire 161u and the lower wire 161d) that bend the bending portion 112 in the UD direction.

The left-right wire drive part 252B is a mechanism that is coupled with the left-right wire attachment and detachment portion 152B and drives the wires (the left wire 161l and the right wire 161r) that bend the bending portion 112 in the LR direction.

The left-right wire drive part 252B has the same structure as the up-down wire drive part 251B, and thus illustration and description thereof will be omitted.

As shown in FIG. 19, the up-down wire drive part 251B includes a support member 255, a wire drive part 256A, an engagement member 258, and an attachment and detachment sensor 259.

The wire drive part 256A is coupled with the rotating drum 156 of the up-down wire attachment and detachment portion 151B, and drives the upper wire 161u and the lower wire 161d. The wire drive part 256A includes a shaft 256a, a motor part 256b, a coupled portion 256c, a torque sensor 256e, and an elastic member 256s.

With the above mechanism, when the up-down wire attachment and detachment portion 151B is mounted in the up-down wire drive part 251B, the wire drive part 256A can drive the upper wire 161u and the lower wire 161d by being interlocked with them.

The drive controller 260B has the same configuration as the drive controller 260 of the first embodiment, and performs bending control on the bending portion 112 based on a bending operation on the bending portion 112 input to the operation device 300 by the operator S.

The drive controller 260B performs angle-free control as in the drive controller 260 of the first embodiment. In Step S130, the drive controller 260B drives the wire 160 until the tension difference between the pair of wires 160 becomes equal to or less than a predetermined threshold value Tth. The bending portion 112 is driven by a wire drive part (an actuator) 250 and bends in a direction to which an external force f_{O} is applied.

In the angle-free control, the drive controller 260B drives the wire 160 by substantially matching an amount of pulling of one of the pair of wires 160 having a higher tension with an amount of sending of the other wire 160 (push-pull control).

According to the electric endoscope system 1000B of the present embodiment, like the electric endoscope system 1000 of the first embodiment, it is possible to appropriately handle bending control (active control) in which the bending portion is actively driven by electric power, and angle-free control (passive control) in which the bending portion is passively driven when an external force is applied. Compared to the electric endoscope system 1000 of the first embodiment, the electric endoscope system 1000B can simplify the configuration of the drive device 200B and the like, thereby reducing costs.

Although the second embodiment of the present invention has been described above in detail with reference to the drawings, the specific configuration is not limited to this embodiment, and design changes may be made within the scope of the gist of the present invention. Moreover, the components shown in the above-described embodiments and modified examples can be configured by appropriately combining them.

### Third Embodiment

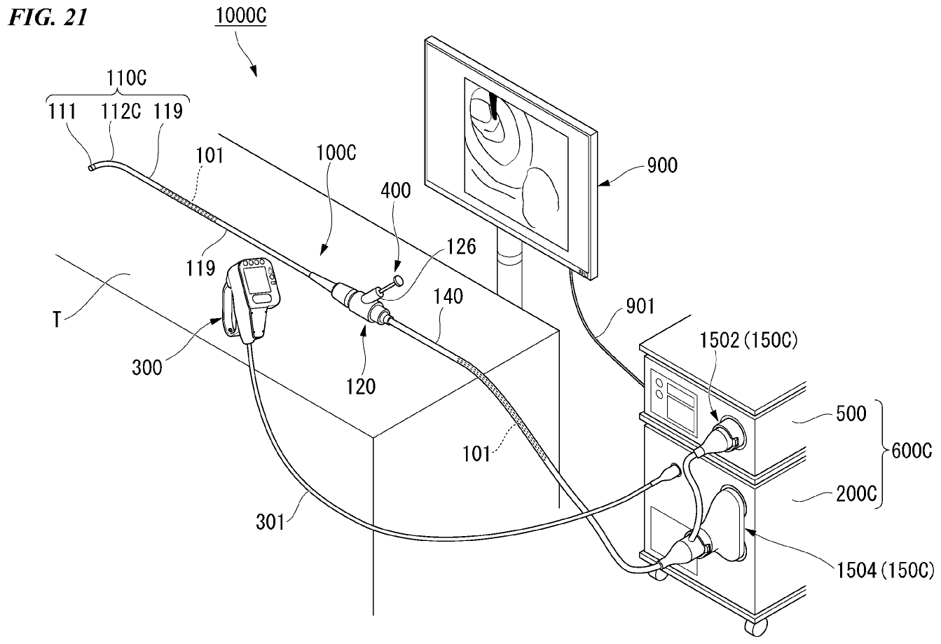

An electric endoscope system 1000C according to a third embodiment of the present invention will be described with reference to FIGS. 21 to 23. In the following description, components that are common to those already described will be designated by the same reference numerals, and redundant description will be omitted.

### Electric Endoscope System 1000C

FIG. 21 is an overall view of the electric endoscope system 1000C according to this embodiment.

As shown in FIG. 21, the electric endoscope system 1000C includes an endoscope 100C, a drive device 200C, an operation device 300, a treatment tool 400, an image control device 500, and a display device 900. The drive device 200C and the image control device 500 constitute a control device 600C that controls the electric endoscope system 1000C.

### Endoscope 100C

The endoscope 100C includes an insertion portion 110C, a connecting portion 120, an extracorporeal flexible portion 140, an attachment and detachment portion 150C, a wire 160C, and a built-in object 170.

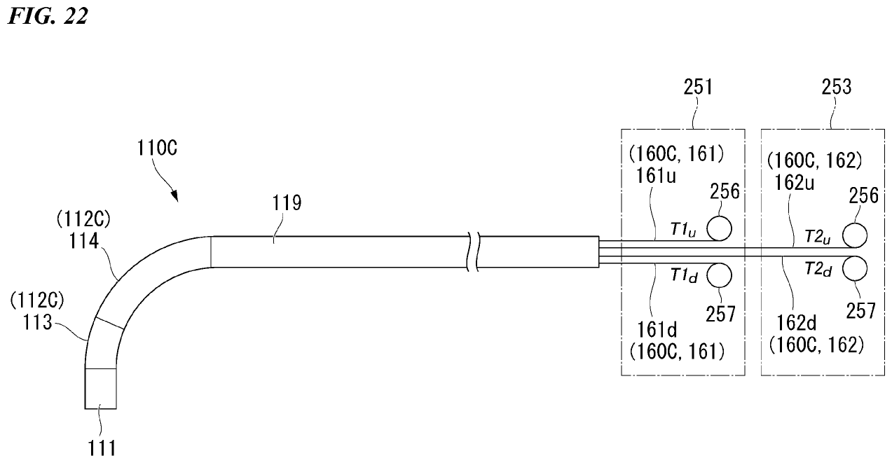

FIG. 22 is a view showing a bending portion 112C.

The insertion portion 110C has a distal end portion 111, a bending portion 112C, and an intracorporeal flexible portion 119. The bending portion 112C includes a first bending portion 113 on the distal end side A1 of the bending portion 112C, a second bending portion 114 on the proximal end side A2 of the bending portion 112C, and an outer sheath 118. The first bending portion 113 and the second bending portion 114 can be bent in different directions.

The wire 160C is a wire that bends the bending portion 112C. The wire 160C includes a first wire 161 that bends the first bending portion 113 and a second wire 162 that bends the second bending portion 114. The first wire 161 and the second wire 162 extend through the internal path 101 to the attachment and detachment portion 150C.

Like the first wire 161, the second wire 162 includes a second upper wire 162u, a second lower wire 162d, a second left wire 162l, and a second right wire 162r.

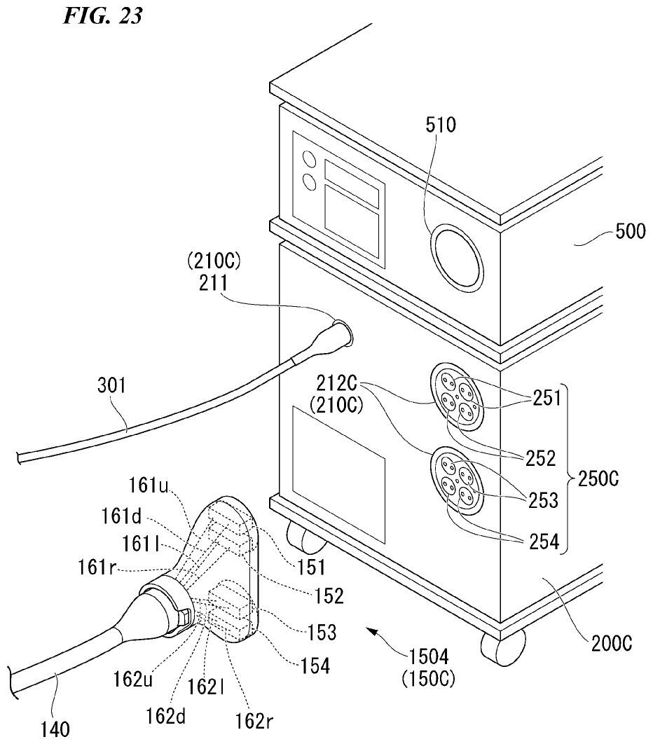

FIG. 23 is a view showing a first attachment and detachment portion 1504 before being mounted in the drive device 200C.

The attachment and detachment portion 150C includes a first attachment and detachment portion 1504 that is mounted in the drive device 200C, and a second attachment and detachment portion 1502 that is mounted in the image control device 500. The first attachment and detachment portion 1504 includes a first up-down wire attachment and detachment portion 151, a first left-right wire attachment and detachment portion 152, a second up-down wire attachment and detachment portion 153, and a second left-right wire attachment and detachment portion 154.

The first up-down wire attachment and detachment portion 151 is a mechanism that detachably connects the wires (the first upper wire 161u and the first lower wire 161d) that bend the first bending portion 113 in the UD direction to the drive device 200C.

The first left-right wire attachment and detachment portion 152 is a mechanism that detachably connects the wires (the first left wire 161l and the first right wire 161r) that bend the first bending portion 113 in the LR direction to the drive device 200C.

The second up-down wire attachment and detachment portion 153 is a mechanism that has the same mechanism as the first up-down wire attachment and detachment portion 151 and detachably connects the wires (the second upper wire 162u and the second lower wire 162d) that bend the second bending portion 114 in the UD direction to the drive device 200C.

The second left-right wire attachment and detachment portion 154 is a mechanism that has the same mechanism as the first left-right wire attachment and detachment portion 152 and detachably connects the wires (the second left wire 162l and second right wire 162r) that bend the second bending portion 114 in the LR direction to the drive device 200C.

### Drive Device 200C

The drive device 200C includes an adapter 210C, an operation receiving part 220, an air supply and suction drive part 230, a wire drive part 250C, and a drive controller 260C.

As shown in FIG. 23, the adapter 210C includes a first adapter 211 and a second adapter 212C. The first adapter 211 is an adapter to which the operation cable 301 is detachably connected. The second adapter 212C is an adapter to which the first attachment and detachment portion 1504 of the endoscope 100C is detachably connected.

The wire drive part 250C is coupled with the first up-down wire attachment and detachment portion 151, the first left-right wire attachment and detachment portion 152, the second up-down wire attachment and detachment portion 153, and the second left-right wire attachment and detachment portion 154 to drive the wire 160C.

As shown in FIG. 23, the wire drive part 250C includes a first up-down wire drive part 251, a first left-right wire drive part 252, a second up-down wire drive part 253, and a second left-right wire drive part 254.

The first up-down wire drive part 251 is a mechanism that is coupled with the first up-down wire attachment and detachment portion 151 and drives the wires (the first upper wire 161u and the first lower wire 161d) that bend the first bending portion 113 in the UD direction.

The first left-right wire drive part 252 is a mechanism that is coupled with the first left-right wire attachment and detachment portion 152 and drives the wires (the first left wire 161l and the first right wire 161r) that bend the first bending portion 113 in the LR direction.

The second up-down wire drive part 253 is a mechanism that has the same mechanism as the first up-down wire drive part 251, is coupled with the second up-down wire attachment and detachment portion 153, and drives the wires (the second upper wire 162u and the second lower wire 162d) that bend the second bending portion 114 in the UD direction.

The second left-right wire drive part 254 is a mechanism that has the same mechanism as the first left-right wire drive part 252, is coupled with the second left-right wire attachment and detachment portion 154, and drives the wires (the second left wire 162l and the second right wire 162r) that bend the second bending portion 114 in the LR direction.

The drive controller 260C has the same configuration as the drive controller 260 of the first embodiment, and performs bending control on the bending portion 112 based on a bending operation on the bending portion 112C input from the operator S to the operation device 300. The drive controller 260C can independently control the first bending portion 113 and the second bending portion 114, and can bend the first bending portion 113 and the second bending portion 114 in different directions (multi-stage bending control).

The drive controller 260C performs angle-free control as in the drive controller 260 of the first embodiment. Specifically, the drive controller 260C performs the angle-free control when the tension difference between a tension T1u of the first upper wire 161u and a tension T1d of the first lower wire 161d becomes larger than a predetermined threshold value Tth, and when the tension difference between a tension T2u of the second upper wire 162u and a tension T2d of the second lower wire 162d becomes larger than the predetermined threshold value Tth.

In the angle-free control, the drive controller 260C drives the wire 160C using either the push-pull control or the antagonistic control, as in the drive controller 260 of the first embodiment.

The drive controller 260C also performs the same angle-free control on the pair of wires 160 (the first left wire 161l and the first right wire 161r) that bend the first bending portion 113 in the left-right direction (the LR direction).

The drive controller 260C performs the same angle-free control on the pair of wires 160 (the second left wire 162l and the second right wire 162r) that bend the second bending portion 114 in the left-right direction (the LR direction).

The angle-free control on the wire 160C may be set and changeable by the operator S. For example, the angle-free control on the first bending portion 113 may be enabled, and the angle-free control on the second bending portion 114 may be disabled on the basis of a predetermined operation input from the operator S to the control button for the angle-free control assigned to the various buttons 352 of the operation device 300. For example, in the angle-free control on the first bending portion 113, the angle-free control on the pair of wires 160 that bend the first bending portion 113 in the up-down direction may be enabled, and the angle-free control for the pair of wires 160 that bend the first bending portion 113 in the left-right direction may be disabled. The operator S can appropriately set the enablement of the angle-free control on the pair of wires 160 according to the type of treatment for the affected area.

Further, the predetermined threshold value Tth may be changed on the basis of a predetermined operation input from the operator S to the control button for the angle-free control assigned to the various buttons 352 of the operation device 300. The operator S can appropriately set the predetermined threshold value Tth according to the type of treatment on the affected area. Furthermore, the predetermined threshold value Tth of the tension difference of the first wire 161 that bends the first bending portion 113 and the predetermined threshold value Tth of the tension difference of the second wire 162 that bends the second bending portion 114 may be set to different threshold values.

According to the electric endoscope system 1000C of the present embodiment, like the electric endoscope system 1000 of the first embodiment, it is possible to appropriately handle bending control (active control) in which the bending portion is actively driven by electric power, and angle-free control (passive control) in which the bending portion is passively driven when an external force is applied.

Although the third embodiment of the present invention has been described above in detail with reference to the drawings, the specific configuration is not limited to this embodiment, and design changes may be made within the scope of the gist of the present invention. Moreover, the components shown in the above-described embodiments and modified examples can be configured by appropriately combining them.

In the above described embodiments, the bending portion was a piece, but the bending portion may include a general joint.

It may be realized by recording a program in each of the embodiments on a computer-readable recording medium, causing a computer system to read and execute the program recorded on the recording medium. The “computer system” includes hardware such as an OS and peripheral devices. Furthermore, the term “computer-readable recording medium” refers to a portable medium such as a flexible disk, a magneto-optical disk, ROM, and CD-ROM, and a storage device such as hard disks built into a computer system. Furthermore, the “computer-readable recording medium” may also include a device that dynamically stores a program for a short period of time, such as a line of communication when a program is transmitted via a network such as the Internet or a communication line such as a telephone line, or a device that stores a program for a predetermined period of time, such as a volatile memory inside a computer system serving as a server or client in that case. Further, the program may be one for realizing a part of the above-described functions, or may be one that can realize the above-described functions in combination with a program already recorded in the computer system.

This invention can be applied to the medical system which observes and treats the inside of a hollow organ, or the like.

What is claimed is:

1. A medical manipulator system comprising:
a medical manipulator having a bending portion and a wire that bends the bending portion;
an actuator that bends the bending portion by driving the wire;
a sensor that detects a tension of the wire; and
a control device that controls the actuator,
wherein the wire includes a pair of first wires that bend the bending portion in a first direction, and
when a tension difference between the pair of first wires is larger than a first threshold value, the control device performs first tension control to drive at least one wire of the pair of first wires so that the tension difference is equal to or less than the first threshold value.

2. The medical manipulator system according to claim 1, further comprising an operation device configured to input an operation instruction for bending the bending portion,
wherein the control device performs the first tension control when bending control is performed on the bending portion on the basis of a bending operation on the bending portion input to the operation device.

3. The medical manipulator system according to claim 1, wherein, when the tension difference between the pair of first wires is larger than the first threshold value, the control device performs the first tension control by sending out one wire of the pair of first wires having a higher tension and pulling the other wire.

4. The medical manipulator system according to claim 1, wherein the actuator has a first actuator that drives one wire of the pair of first wires, and a second actuator that drives the other wire, and
when the tension difference is equal to or larger than the first threshold value, the control device performs the first tension control by sending out one of the pair of first wires having a higher tension and keeping the tension of the other wire constant.

5. The medical manipulator system according to claim 1, wherein the control device performs the first tension control so that an operation speed of the bending portion becomes slower as the tension difference becomes smaller.

6. The medical manipulator system according to claim 1, wherein the wire includes a pair of second wires that bend the bending portion in a second direction different from the first direction, and
when a tension difference between the pair of second wires is larger than a second threshold value, the control device performs second tension control to drive at least one wire of the pair of second wires so that the tension difference is equal to or less than the second threshold value.

7. The medical manipulator system according to claim 6, further comprising an operation device configured to input an operation instruction for bending the bending portion,
wherein the control device is configured to disable at least one of the first tension control and the second tension control on the basis of a predetermined operation input input to the operation device.

8. The medical manipulator system according to claim 6, further comprising an operation device configured to input an operation instruction for bending the bending portion,
wherein the control device is configured to change at least one of the first threshold value and the second threshold value on the basis of a predetermined operation input input to the operation device.

9. The medical manipulator system according to claim 6, wherein
the bending portion has a first bending portion and a second bending portion,
the pair of first wires bend the first bending portion in the first direction,
the wire includes a pair of third wires that bend the second bending portion in the first direction, and
when a tension difference between the pair of third wires is larger than a third threshold value, the control device performs third tension control to drive at least one wire of the pair of third wires so that the tension difference is equal to or less than the third threshold value.

10. The medical manipulator system according to claim 9, wherein
the wire includes a pair of fourth wires that bend the second bending portion in the second direction, and
when a tension difference between the pair of fourth wires is larger than a fourth threshold value, the control device performs fourth tension control to drive at least one wire of the pair of fourth wires so that the tension difference is equal to or less than the fourth threshold value.

11. The medical manipulator system according to claim 10, further comprising an operation device configured to input an operation instruction for bending the bending portion,
wherein the control device is configured to disable at least one of the first tension control, the second tension control, the third tension control, and the fourth tension control on the basis of a predetermined operation input input to the operation device.

12. The medical manipulator system according to claim 10, further comprising an operation device configured to input an operation instruction for bending the bending portion,
wherein the control device is configured to change at least one of the first threshold value, the second threshold value, the third threshold value, and the fourth threshold value on the basis of a predetermined operation input input to the operation device.

13. A method for controlling a medical manipulator, which controls a bending motion of the medical manipulator, comprising:
performing first tension control to drive at least one of a pair of first wires so that a tension difference between the pair of first wires that bend the medical manipulator in a first direction is equal to or less than a first threshold value when the tension difference is larger than the first threshold value.

14. The method for controlling a medical manipulator according to claim 13, wherein first tension control is performed by sending out one of the pair of first wires having a higher tension and pulling the other wire when the tension difference between the pair of first wires is larger than the first threshold value.

15. The method for controlling a medical manipulator according to claim 13, wherein, when the tension difference is equal to or larger than the first threshold value, the first tension control is performed by sending out one of the pair of first wires having a higher tension and keeping the tension of the other wire constant.

16. The method for controlling a medical manipulator according to claim 13, wherein the first tension control is performed so that a bending operation speed of the medical manipulator becomes slower as the tension difference becomes smaller.

17. A control device which controls a bending operation of a medical manipulator, wherein the control device is configured to output a control instruction for a pair of first wires that bend the medical manipulator in a first direction, and performs first tension control to drive at least one wire of the pair of first wires so that a tension difference between the pair of first wires is equal to or less than a first threshold value when the tension difference is larger than the first threshold value.

18. The control device according to claim 17, wherein the control device performs the first tension control even when an operation input is received and bending of the medical manipulator is controlled on the basis of the operation input.

19. The control device according to claim 17, wherein, when the tension difference between the pair of first wires is larger than the first threshold value, the control device performs the first tension control by sending out one of the pair of first wires having a higher tension and pulling the other wire.

20. The control device according to claim 17, wherein the control device is configured to control a first actuator connected to one wire of the pair of first wires and a second actuator connected to the other wire, and performs the first tension control by sending out one of the pair of first wires having a higher tension and keeping the tension of the other wire constant when the tension difference is equal to or larger than the first threshold value.
